(19) United States
(12) Patent Application Publication
(10) Pub. No.: US 2009/0036738 A1
(43) Pub. Date: Feb. 5, 2009

(54) Device for creating a free transcutaneous access to an endoscopic operating area

(75) Inventors: Alfred Cuschieri, St. Andrews (GB); Timothy Graham Frank, Wormit Newport-On-Tay (GB); Stuart I. Brown, St. Andrews (GB); James Gove, GB-Dundee (GB); Leslie Kelly, Cupar (GB); Duncan J. Martin, GB-Dundee (GB); Ian Rutherford, GB-Dundee (GB)

Correspondence Address: ST. ONGE STEWARD JOHNSTON & REENS, LLC, 986 BEDFORD STREET, STAMFORD, CT, 06905-5619, US

(73) Assignee: University Of Dundee; Great Britain

(21) Appl. No.: 11/450,824

(22) Filed: Jun. 9, 2006

(30) Foreign Application Priority Data
Jun. 9, 2005 (DE) 10 2005 026 467.0

(51) Int. Cl. A61B 1/00

(52) U.S. Cl. 600/114

(57) ABSTRACT

The invention relates to a device for creating a free transcutaneous access to an endoscopic operating area, in particular to a hollow internal body organ, which device has a conical inset that is to be inserted into an incision. A device for creating a transcutaneous access that combines simple construction with good handling qualities for several medical instruments is, according to the invention, characterized in that several medical instruments can be inserted simultaneously into the operating area by way of the conical inset, so that the individual medical instruments are positioned in the conical inset at a secured angle to one another.

### FIELD OF THE INVENTION

The invention relates to a device for creating a free transcutaneous access to an endoscopic operating area, in particular to a hollow internal body organ, which device has a conical inset that can be inserted into an incision.

### BACKGROUND OF THE INVENTION

In minimally invasive surgery, transcutaneous accesses to operating areas are provided as a rule by means of so-called trocars. Trocars consist of a rigid metallic housing and the trocar triblet that can be inserted into the trocar housing and constitutes the actual trocar, which has a sharp point for forming an incision. After forming the incision, the trocar housing is pushed into the patient's body and the trocar triblet is removed from the trocar housing. Subsequently a surgeon can introduce through the trocar housing surgical instruments such as endoscopes and other surgical operating implements.

In hollow internal organs, such as the stomach, even during anesthesia relative motions occur over a certain area between the abdominal surface and the hollow internal organ, so that here it is necessary to employ relatively long trocar housings to ensure that the trocar housing does not slip out of the hollow organ because of the motions of the hollow organ.

A generic device for creating a free transcutaneous access to an endoscopic operating area is known, for instance, from U.S. Pat. No. 5,634,937 A. The disadvantage of this device is that only one medical instrument at a time can be introduced into the operating area through the conical inset.

From DE 199 16 088 A1, another device for creating a transcutaneous access to a hollow internal body organ is known. This familiar device serving for endoluminal operations consists of a foot portion, which can be secured by clamping to a hollow probe, which is guided outward from the body interior through the incisions in the wall of the hollow organ and in the abdominal surface. A tubular-shaped shaft can be connected to this foot portion, secured in this manner on the incision site by means of a coupling element, and said shaft outside the body extends the hollow probe in such a way that medical instruments, for instance endoscopes and other operating implements, can be introduced into the hollow organ by way of the hollow shaft and the probe.

This familiar device has proven itself thorough in practice; however, it is, for one thing, relatively expense to produce and, in addition, the free threshold of the hollow shaft and the probe are not of such dimensions that an endoscope and other instruments can be introduced into the operating area simultaneously, and thus in the customary working angle of 30 degrees to one another, by means of this device.

It is consequently the aim of the invention to produce a device for creating a free transcutaneous access, which device is of simple construction and offers good maneuverability for several medical instruments.

### SUMMARY OF THE INVENTION

The means of achieving this aim, according to the invention, is characterized in that several medical instruments can be introduced simultaneously into the operating areas by way of the conical inset, so that the individual medical implements are positioned at a determined angle to one another in the conical inset.

Thanks to the configuration of the device according to the invention, it is possible for the surgeon simultaneously to introduce several implements into the operating area by way of a conical inset, so that thanks to the determined angle of the medical implements to one another, an exact guidance of the instruments is ensured in the operating area.

It is further proposed with the invention that on the inside of the inset at least one guidance device for a medical instrument, in particular an endoscope, is configured. By providing this guidance device, which can be configured, for instance, as a guide channel running in the conic direction of the inset, the surgeon can be considerably relieved because he or she no longer needs to use one hand to secure, during the entire operation, the instrument positioned in this guidance device, such as an endoscope. The guidance devices furthermore guarantee that the medical instruments are held at a firmly predetermined angle to one another.

The conic angle (alpha) spread apart by the inner wall of the inset is advantageously between 30 and 90 degrees, and preferably 60 degrees. This preferred conic angle of the inset according to this invention ensures that the angle of 30 degrees desired in practice can be maintained between the optic axis of the endoscope and the other instruments without problems, even with two instruments present.

According to a practical embodiment of the invention, it is proposed that the conic inset is of blunted conic form configured in such a way that the inset has an aperture on the distal side smaller in diameter than on the proximal side.

To prevent the inset according to this invention from slipping out of the incision, it is proposed with the invention that the conic inset can be secured in the incision. For this purpose, according to a preferred embodiment of the invention, it is proposed that on the exterior of the inset, which preferably consists of a rigid plastic, at least one securing element is positioned.

According to a first practical embodiment of the invention, it is proposed that the at least one securing element takes the form of a screw-in socket, which is at least single-threaded and is mounted on the exterior of the inset, and by means of which the inset can be screwed into the incision.

According to a second embodiment according to the invention for configuring the at least one securing element, it is proposed that the at least one securing element is configured as a surrounding click-stop ridge. At least two surrounding click-stop ridges are advantageously configured, positioned parallel to one another, on the exterior of the inset as securing elements. By means of series of click-stop ridges, it is possible to vary insertion depth of the inset into the incision or to adjust it flexibly to the specific operating conditions.

To facilitate insertion of the conical inset into the incision and to be able to tilt the inset, once inserted in the incision, with respect to its longitudinal axis, it is proposed by the invention that a guide arm can be secured on the inset. For this purpose, a coupling element for securing the guide arm is advantageously positioned on the exterior of the inset.

Finally, it is proposed with the invention that at least the aperture on the proximal side of the inset can be closed by means of a covering element in order to insulate the operating area hygienically from its surroundings.

According to a first embodiment of the invention, the covering element is configured as a membrane that can be penetrated by the medical implements that are to be inserted into the inset, in such a way that the membrane can be configured for instance as an insulating disc made of a silicon material.

It is proposed with a second embodiment of the invention that the covering element is configured as a cap equipped with at least one aperture for receiving a medical implement that is to be inserted into the conical inset, in such a way that every aperture in the cap is provided with an elastic insulating membrane, which provides exterior insulation for the instrument shaft that is to be inserted.

To facilitate precisely aimed insertion of the medical implements into the operating area, the cap according to the invention can be secured onto the conical inset secure against rotation in such a way that, when it is locked, at least one aperture in the cap is aligned with a guide device positioned on the interior of the conical inset. A medical implement inserted into the cap aperture is then guided to the operating area by means of the guide device with exact positioning. The cap advantageously is made of a transparent material, in particular a plastic material, so that it is possible to determine through the transparent cap whether the inserted medical implements are properly seated in the guide devices on the interior of the conical inset.

The cap is insulated from the conical inset, according to the invention, by means of an insulating element as an intermediate layer.

Additional characteristics and advantages of the invention can be seen from the appended illustrations, which present merely a schematic depiction of an embodiment of a device according to this invention for creating a free transcutaneous access to an endoscopic operating area.

### BRIEF DESCRIPTION OF THE DRAWINGS

FIG. 1 shows a schematic depiction of an operation with a device according to the invention.

FIG. 2 shows a perspective side view of a device according to the invention.

FIG. 3 shows an overhead view of the device according to FIG. 2.

FIG. 4 shows a schematic sectional view depicting a device according to the invention inserted into the abdominal covering and the abdominal wall.

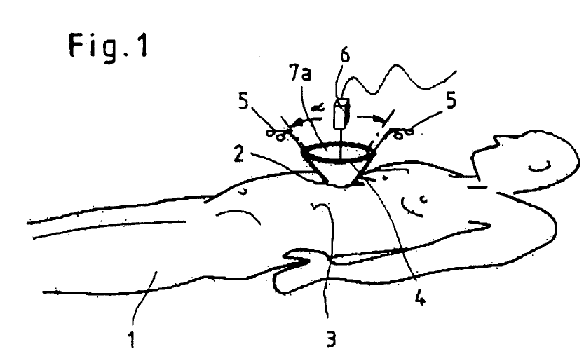

In FIG. 1 is seen an exemplary schematic depiction of an endoscopic operation. At the start of the operation an incision 2 is made in the abdominal covering 3 of the patient 2, for instance by means of a trocar triblet. Thereafter a conical inset 4 is inserted into this incision 2, which inset 4 allows a free transcutaneous access for inserting medical implements, such as cutting and/or gripping instruments 5 and endoscopes or video cameras 6, into the operating area.

### DETAILED DESCRIPTION OF THE INVENTION

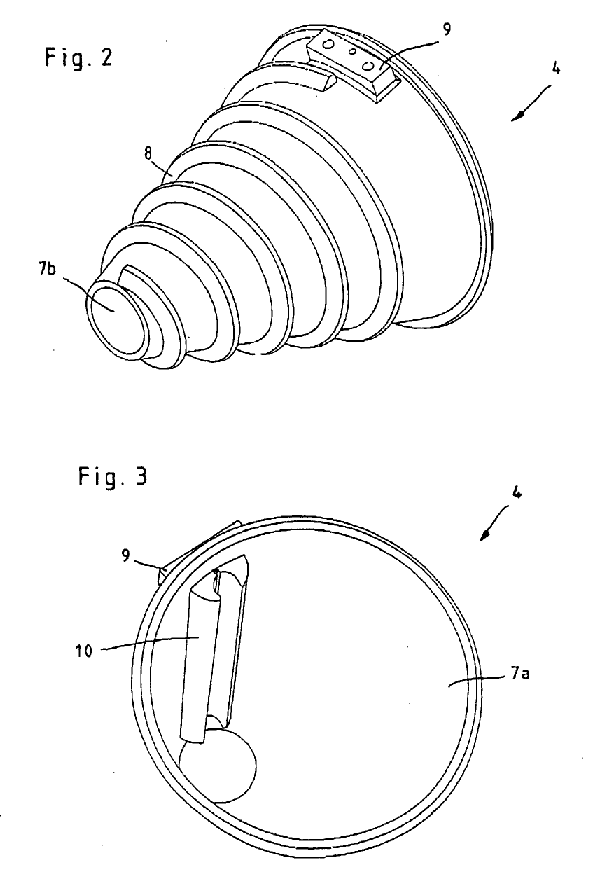

The structure of the conical inset 4 can be seen more exactly from the depictions in FIGS. 2 and 3. As shown in particular in FIG. 2, the conical inset 4, which consists preferably of a rigid plastic material, is configured in the illustrated embodiment as a blunted conical shape, in such a way that the inset 4 has an aperture 7a that is smaller in diameter on the distal side than on the proximal side.

The conical angle alpha, stretched apart by the conical inner surface of the inset 4, is preferably 60 degrees. This angles is particularly advantageous because in this embodiment, as can be seen from FIG. 1, at least two cutting and/or gripping instruments 5 along with an endoscope or a video camera 6 can be introduced into the operating area by the conical inset 4 in such a way that the angle between the optical axis of the endoscope or video camera 6 and the longitudinal axis of the cutting and/or gripping instruments 5 can be 30 degrees, as desired in practice. This angle position makes possible for the surgeon a position between the optical tool and the actual working implements that otherwise is possible only by using at least two incisions 2.

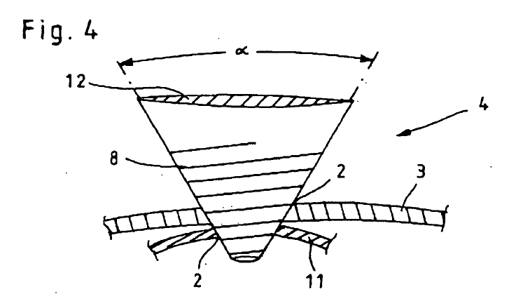

To be able to secure the conical inset 4 in the incision 2 and, thereby to prevent the inset 4 from slipping out of the incision 2, in the illustrated embodiment a securing element is positioned on the exterior of the inset 4 configured as a screw track 8. By means of this screw track 8, which can also consist of several parallel screw tracks 8, the conical inset 4 can be easily and quickly screwed into the incision 2, as is shown in FIG. 4.

Alternatively to the illustrated embodiment, it is also possible, for instance, to configure the securing element as a surrounding click-stop ridge positioned on the exterior of the inset 4, in such a way that is advantageous with this embodiment to use several click-stop ridges arranged parallel to one another, in order to vary the insertion depth of the inset 4 into the incision 2 or to be able to adapt flexibly to the specific operating conditions.

It is possible to facilitate insertion or screwing of the conical inset 4 into the incision 2 if a guide arm, pointing radially outward, can be secured on the inset 4. In the embodiment shown in FIG. 2, the guide arm as such is not depicted, but rather a coupling element 9, positioned on the outside of the inset 4 to secure the guide arm. The guide arm, moreover, can be used to tilt the inset 4, which happens to be in the operating position in the incision 2, as a whole relative to the abdominal surface 3 of the patient 1.

The overhead view of the conical inset 4 of FIG. 3 shows that in the illustrated embodiment a guide device 10 configured as a guide channel running in the conical direction of the inset 4 is positioned on the interior of the inset 4. Thanks to the presence of this guide device 10, the surgeon can be relieved in that he no longer needs to hold firmly with one hand the instrument, such as an endoscope, positioned in this guide device 10 throughout the entire length of the operation.

FIG. 4, finally, shows the use of a conical inset 4 as previously described in an endoluminal operation on a hollow internal body organ, in this case the stomach. In this operation the conical inset 4 is inserted into incisions 2 in the abdominal covering 3 and in the stomach wall 11 of the patient 1, so that the operator has at his disposal a direct and easily produced transcutaneous access to the operating area, which allows the surgeon extensive free play for maneuvering the medical surgical implement. The sectional drawing in FIG. 4 clearly shows how the screwing track 8, serving as a securing element, on the exterior of the conical inset 4 grips the individual tissue parts, specifically the abdominal covering 3 on the one hand and the stomach wall 11 on the other, and thus prevents the conical inset 4 from accidentally slipping out of the incisions 2.

As can further be seen from FIG. 4, at least the aperture 7b on the proximal side of the conical inset 4 can be closed by a covering element 12 in order to insulate the operating area hygienically from its surroundings. In the illustrated embodiment, the covering element 12 is configured as a membrane, made of an elastic material that can also be penetrated by the implements that are to be inserted into the conical inset 4, so that the membrane, even when operating implements are inserted, provides insulation in direct contact for the particular instrument shafts. Appropriate material for constructing the self-closing and self-insulating covering membrane could be, for instance, a silicon material.

As an alternative to the illustrated configuration of the covering element 12 as a membrane, it is also possible of course to configure the covering element 12 as a cap that can be secured on the conical inset 4, in which cap at least one aperture is formed for inserting a medical instrument that is to go into the conical inset and where every aperture in the cap is equipped with an elastic insulating membrane, which insulates the inserted instrument shaft against the exterior.

To facilitate precisely aimed insertion of the medical instrument into the operating area, the cap can be secured on the conical inset 4 secure against rotation in such a way that when it is closed at least one aperture in the cap is aligned with a guide device 10 positioned on the inside of the conical inset 4. A medical implement inserted into the cap aperture is then guided by the guide device 10 and precisely positioned in the operating area. Thanks to the cap, made of a transparent material, in particular a plastic material, in this manner it is possible to ascertain from outside whether the inserted medical implement has been properly seated in the guide devices 10 on the inside of the conical inset 4.

The handling of the previously described device for creating a transcutaneous access to an endoscopic operating area is as follows.

At the start of the operation, by means of a pointed trocar triblet an incision 2 is made in the abdominal covering 3 of the patient 1. In an endoluminal operation a second incision 2 is made by means of the trocar triblet in the outer wall of the hollow interior body organ. In an endoluminal stomach operation the incision is produced in the stomach wall advantageously while controlling and guiding a gastroscope that is inserted into the stomach by way of the digestive tract.

After the incision 2 or incisions 2 are made, the conical inset 4 is placed on the trocar triblet as a guide and, by means of the screwing track 8, it is screwed into the incision or incisions 8. The screwing track 8 serving as a securing element holds the conical inset 4 securely and in an exact position in the body of the patient, so that the trocar triblet can then be withdrawn from the incision or incisions 2 again.

Now the surgeon has an easily accessible transcutaneous access to the operating area. Because of the great conical angle alpha of the conical inset 4, several surgical instruments can be introduced into the operating area simultaneously by way of the only one transcutaneous access, so that it is possible to maintain the 30 degree advantageous optical angle of approach of the working instruments to the optical axis of a viewing instrument. The good accessibility of the operating area, moreover, facilitates the removal of severed tissue from the body and/or the insertion of sewing material, for instance, into the body.

On completion of the operation, the inset 4 is again unscrewed out of the incision 2 and the incision is sewn up. In the endoluminal operation illustrated in FIG. 4 the conical inset 4 is at first only screwed out of the incision 2 in the stomach wall 11, in order thereafter to close up the incision 2 in the stomach wall 11 by means of stitching.

A device of the aforementioned configured for creating a transcutaneous access to an endoscopic operating area is distinguished in that it combines an especially simple construction with the greatest possible freedom of motion for the surgeon during the operation.

For the patient 1, the use of the conical inset 4 is advantageous because as a rule it makes it unnecessary to make a second or third incision 2 for introducing additional surgical implements.

1. A device for creating a free transcutaneous access to an endoscopic operating area, in particular to a hollow internal body organ, which devices has a conical inset that can be inserted into an incision, characterized in that several medical implements can be inserted simultaneously into the operating area by way of the conical inset, so that the individual medical implements are positioned at a secured angle to one another in the conical inset.

2. A device according to claim 1, characterized in that on the interior of the conical inset at least one guide device is configured for a medical instrument, in particular an endoscope.

3. A device according to claim 2, characterized in that the guide device is configured as a guide channel running in the conical direction of the inset.

4. A device according to claim 1, characterized in that the conical angle (alpha) stretched apart by the conical inside surface of the inset is 30 to 90 degrees, preferably 60 degrees.

5. A device according to claim 1, characterized in that the conical inset is configured as a blunted conical form in such a way that the inset has an aperture with a smaller diameter on the distal side than on the proximal side.

6. A device according to claim 1, characterized in that the conical inset is made of a basically rigid plastic material.

7. A device according to any claim 1, characterized in that the conical inset can be secured in the at least one incision.

8. A device according to claim 7, characterized in that at least one securing element is positioned on the exterior of the conical inset.

9. A device according to claim 8, characterized in that at the at least one securing element is configured as a screwing track that is at least single-threaded and positioned on the exterior of the conical inset.

10. A device according to claim 8, characterized in that the at least one securing element is configured as a surrounding click-stop ridge positioned on the exterior of the conical inset.

11. A device according to claim 10, characterized in that at least two surrounding click-stop ridges are configured as securing elements running parallel to one another on the exterior of the conical inset.

12. A device according to claim 1, characterized in that at a guide arm can be secured on the conical inset.

13. A device according to claim 12, characterized in that a coupling element for securing the guide arm is positioned on the exterior of the conical inset.

14. A device according to claim 1, characterized in that at least the proximal-side aperture of the conical inset can be closed by a covering element.

15. A device according to claim 14, characterized in that the covering element is configured as a membrane.

16. A device according to claim 15, characterized in that the membrane is configured as a membrane, made for instance of a silicon material, that can be penetrated by the medical instruments that are to be inserted into the conical inset.

17. A device according to claim 14, characterized in that the covering element is configured as a cap equipped with at least one aperture for inserting a medical instrument that is to be inserted into the conical inset, so that every aperture in the cap is equipped with an elastic insulating membrane.

18. A device according to claim 17, characterized in that the cap can be secured on the conical inset secure against rotation, in such a way that in the closed position at least one aperture in the cap is aligned with a guide device positioned on the inside of the conical inset.

19. A device according to claim 16, characterized in that the cap is made of a transparent material, in particular a plastic material.

20. A device according to claim 16, characterized in that the cap is insulated by an insulating element against the conical inset.
